(19) United States
(12) Patent Application Publication
(10) Pub. No.: US 2012/0114757 A1
(43) Pub. Date: May 10, 2012

(54) PARTICLES COMPRISING DROSPIRENONE ENCAPSULATED IN A POLYMER

(75) Inventors: Sascha General, Berlin (DE); Filip Novak, Berlin (DE)

(73) Assignee: BAYER PHARMA AKTIENGESELLSCHAFT, Berlin (DE)

(21) Appl. No.: 13/202,322

(22) PCT Filed: Feb. 12, 2010
(86) PCT No.: PCT/EP10/51759

§ 371 Date: Oct. 25, 2011

(30) Foreign Application Priority Data
Feb. 18, 2009 (EP) 09002258.3

(51) Int. Cl. A61K 9/14; A61P 5/30; A61P 15/00; A61K 31/585; A61P 15/18

(52) U.S. Cl. 424/489; 514/173; 514/170

(57) ABSTRACT

The present invention relates to particles comprising Drospirenone encapsulated in a polymer selected from the group consisting of glycolic acid polymer, lactic acid polymer, poly caprolactones, poly anhydrides and any copolymer of these, e.g., poly(lactic acid-co-glycolic acid) polymer and any combination of these. Furthermore, the present invention also relates to compositions comprising such particles. The present invention also relates to the use of such particles or compositions as contraceptives and for treatment of diseases, disorders and symptoms associated with deficient endogenous levels of estrogen in women.

### TECHNICAL FIELD OF THE INVENTION

The present invention relates to particles comprising Drospirenone encapsulated in a polymer selected from the group consisting of glycolic acid polymer, lactic acid polymer, poly(lactic acid-co-glycolic acid) polymer and any combination of these. Furthermore, the present invention also relates to compositions comprising such particles.

The present invention also relates to the use of such particles or compositions as contraceptives and for treatment of diseases, disorders and symptoms associated with deficient endogenous levels of estrogen in women.

### BACKGROUND OF THE INVENTION

Drospirenone is a progestin which is known to be used in contraceptives and for treatment of diseases, disorders and symptoms associated with deficient endogenous levels of estrogen in women.

Today different formulations for administration of steroid hormones in general are known which each offer different advantages and disadvantages.

Besides the formulations efficiency the consumers experience with the ease of using said formulations is also relevant especially as correct use is necessary for the product to work satisfactory.

Aspects which are relevant when designing such formulations are among other things the mode of administration and frequency of administration.

Drospirenone has certain advantages compared to other progestins such as a positive effect on skin appearance and an ability to lessen complications relating to premenstrual syndrome.

However, for Drospirenone to be efficient it is typically administered at higher doses than other progestins. This poses a challenge when Drospirenone is designed for injection due to the limitation of administration volume. Furthermore Drospirenone isomerizes at acidic pH resulting in an inactivation of the compound. There is an ongoing need for developing steroid hormone formulations so as to suit the individual consumer's needs.

One advantage of the present invention is that it results in a depot effect enabling a less than daily administration frequency.

WO 93/00070 relates to sustained release formulations for maintaining low serum levels of an androgen. WO 93/00070 describes microencapsulation of an androgen in a biocompatible, biodegradable polymer, e.g. poly(d,1-lactide-co-glycolide) and a long list of suitable androgens is given. However, WO 93/00070 does not disclose Drospirenone.

U.S. Pat. No. 3,733,919 discloses formulations of polylactide and drug which provide a slow sustained release of the drug over a controlled period of time.

EP 0 058 481 discloses pharmaceutical compositions comprising a polylactide and a pharmacologically active, acid stable polypeptide. EP 0 058 481 further discloses the composition of that invention are not suitable for polypeptides which are not stable under acid conditions.

As described above Drospirenone isomerizes at acidic pH in aqueous media creating a therapeutical inactive isomer. It was therefore a surprise when the inventors of the present invention found encapsulation of Drospirenone in polymers of lactic and/or glycolic acid monomers did not result in a notable degradation of Drospirenone as such polymers are known from e.g. EP 0 058 481 to create an acidic environment. Other biodegradable polymers that create acidic environment are poly caprolactones and poly anhydrides. These classes of polymers hydrolize in aqueous media, similar to polymers of lactic or glycolic acid residues, generating free ionisable acidic functions, e.g. carboxylic acid residues.

### SUMMARY OF THE INVENTION

In a first aspect the present invention relates to particles comprising Drospirenone encapsulated in a polymer selected from the group consisting of polyglycolic acid, polylactic acid, poly caprolactones, poly anhydrides and any copolymer of these. In particular copolymers of glycolic acid and lactic acid.

In a further aspect the present invention relates to a composition comprising particles according to the present invention.

In an even further aspect the present invention relates to a method of producing particles according to present invention comprising the steps of:
a) mixing Drospirenone and a polymer selected from the group consisting of polyglycolic acid, polylactic acid, poly caprolactones, poly anhydrides and any copolymer of these, in a solvent
b) adding a hydrophilic polymer to the mixture obtained from step a)
c) emulsifying or homogenising the mixture obtained from step b), and
d) evaporating the solvent of step a).

In an even further aspect the present invention relates to particles of the present invention or a composition according to the present invention for the use as a medicament.

In an even further aspect the present invention relates to a method for inhibition of ovulation in a women comprising subcutaneous injection of particles according to the present invention or a composition according to the present invention.

In an even further aspect the present invention also relates to a method for the treatment of diseases, disorders and symptoms associated with deficient endogenous levels of estrogen in a women comprising subcutaneous injection of the particles or a composition according to the present invention.

### BRIEF DESCRIPTION OF THE FIGURES

FIG. 1 shows the shape and the surface of the micro particles as visualised with a light microscope and produced with emulsion 1 in example 1.

FIG. 2 shows the shape and the surface of the micro particles visualised with a light microscope and produced with emulsion 8 in example 1

FIG. 3 shows the in vitro release of Drospirenone from micro particles produced with emulsions 1-4.

FIG. 4 shows the in vitro release of Drospirenone from micro particles produced with emulsions 5-8.

FIG. 5 shows the mean concentration of Drospirenone found in the serum (measured in ng/ml) of a rat after a single subcutaneous (s.c.) injection of different formulations of Drospirenone into a rat.

The present invention will now be described in more detail in the following.

### DETAILED DESCRIPTION OF THE INVENTION

The present invention relates to particles comprising Drospirenone encapsulated in a polymer selected from the group consisting of polyglycolic acid, polylactic acid, poly caprolactones, poly anhydrides and any copolymer of these, e.g. a copolymer of glycolic acid and lactic acid. In the context of the present invention the term “any copolymer of these” refers to copolymers comprising one or more of the monomers present in the other polymers of the list.

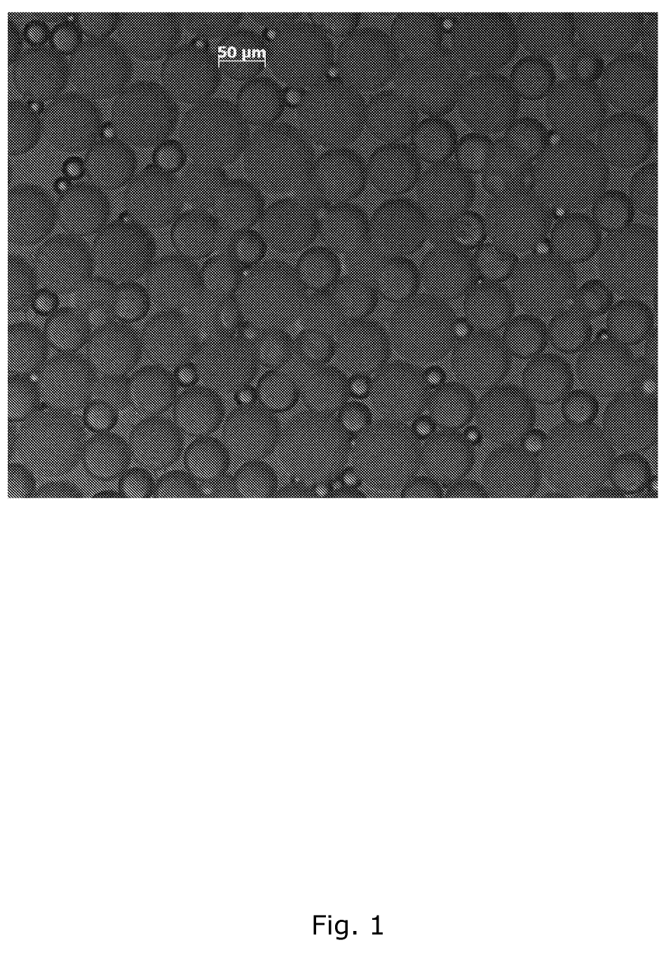

An advantage of the present invention is that the pharmacokinetic profile of particles comprising Drospirenone according to the present invention is a long-term steady state release of Drospirenone (see example 4). Furthermore, it is an advantage that compared to a formulation of Drospirenone in water a large initial release of Drospirenone, i.e. a so called initial burst, is avoided (as shown in example 2 and FIG. 1).

### Particles

In the context of the present invention the term “particle” or “particles” is used according to the general understanding of this term. Thus the particle of the present invention may be spherical, substantially spherical, or non-spherical, such as irregularly shaped particles or ellipsoidally shaped particles.

When particles, such as those according to the present invention, are produced this typically result in the production of particles of various sizes as it is difficult from a manufacturing point of view to ensure that all particles are of the same size. To reflect this variation the size of the particles are often described by the particle size distribution denoted dₓ.

When used herein, the term “dₓ particle size” is intended to mean that the particle size distribution is so that at least X% of the particles has a particle diameter of less than the specified value, calculated from the volume distribution curve under the presumption of spherical particles.

Therefore, it is important to note that whenever the terms “particle size”, “particle size distribution”, “particle diameter”, “d₉₀”, “d₅₀”, etc., are used herein it should be understood that the specific values or ranges used in connection therewith are always meant to be determined from the volume distribution curve under the presumption of spherical particles. The particle size distribution may be determined by various techniques, e.g. laser diffraction, and will be known to the person skilled in the art.

In a particular embodiment the d₅₀ of the particles of the present invention are in the range of 1-300 μm, such as in the range of 10-200 μm, or in the range of 10-150 μm, or in the range of 20-100 μm, or in the range 30-70 μm.

The term “encapsulated” means in the present context that Drospirenone is homogeneous distributed in the polymer matrix of the particles. Such homogeneous distribution of a drug in polymer particles is known as a matrix encapsulation. However, due to the manufacturing process it is foreseen that minor amounts of Drospirenone may also be present on the outside of the particle and/or mixed with the polymer making up the shell of the particle.

Drospirenone is a synthetic progestin which is an analog of spironolactone, and it has a molecular weight of 366.5 and the molecular formula C₂₄H₃₀O₃. It is also described with the chemical formula 6β,7β;15β,16β-dimethylene-3-oxo-17α-pregn-4-ene-21,17-carbolactone.

Apart form the active substance itself, it is envisaged that an ester or prodrug of drospirenone may be employed in the present composition, e.g. an oxyiminopregnane carbolactone as disclosed in WO 98/24801.

Drospirenone, which may be prepared substantially as described in, e.g., U.S. Pat. No. 4,129,564, or WO 98/06738, is a sparingly soluble substance in water and aqueous buffers at various pH values. Furthermore, drospirenone is rearranged to an inactive isomer under acid conditions. To ensure good bioavailability of the compound, it is therefore advantageously provided in a form that promotes rapid dissolution thereof.

The formulations of the present invention may in particular be used as contraceptives and for treatment of diseases, disorders and symptoms associated with deficient endogenous levels of estrogen in women.

Hence in a preferred embodiment the formulations of the present invention may further comprise an estrogen, natural or a synthetic derivative thereof. Furthermore, it is envisioned that such an estrogen may be encapsulated in a particle of the present invention in combination with Drospirenone. However, it is also envisioned that the estrogen may be provided in another formulation than the particles of the present invention which then may or may not be mixed or combined with the particles of the present invention prior to administration.

Hence in one embodiment the particles of the present invention further comprises an estrogen.

If the particles are used as a contraceptive said estrogen may in particular be ethinylestradiol.

If the particles are used for treatment of diseases, disorders and symptoms associated with deficient endogenous levels of estrogen in women said estrogen may in particular be selected from the group consisting of estradiol, estradiol sulfamates, estradiol valerate, estradiol benzoate, ethinyl estradiol, estrone, estriol, estriol succinate and conjugated estrogens, including conjugated equine estrogens such as estrone sulfate, 17β-estradiol sulfate, 17α-estradiol sulfate, equilin sulfate, 17β-dihydroequilin sulfate, 17α-dihydroequilin sulfate, equilenin sulfate, 17β-dihydroequilenin sulfate and 17α-dihydroequilenin sulfate or mixtures thereof. Particularly interesting estrogens are selected from the group consisting of estradiol, estradiol sulfamates, estradiol valerate, estradiol benzoate, estrone, and estrone sulfate or mixtures thereof, notably estradiol, estradiol valerate, estradiol benzoate and estradiol sulfamates. Most preferred is estradiol or estradiol sulfamates, particularly estradiol.

In certain embodiments of the invention, the particles may comprise more than one estrogen.

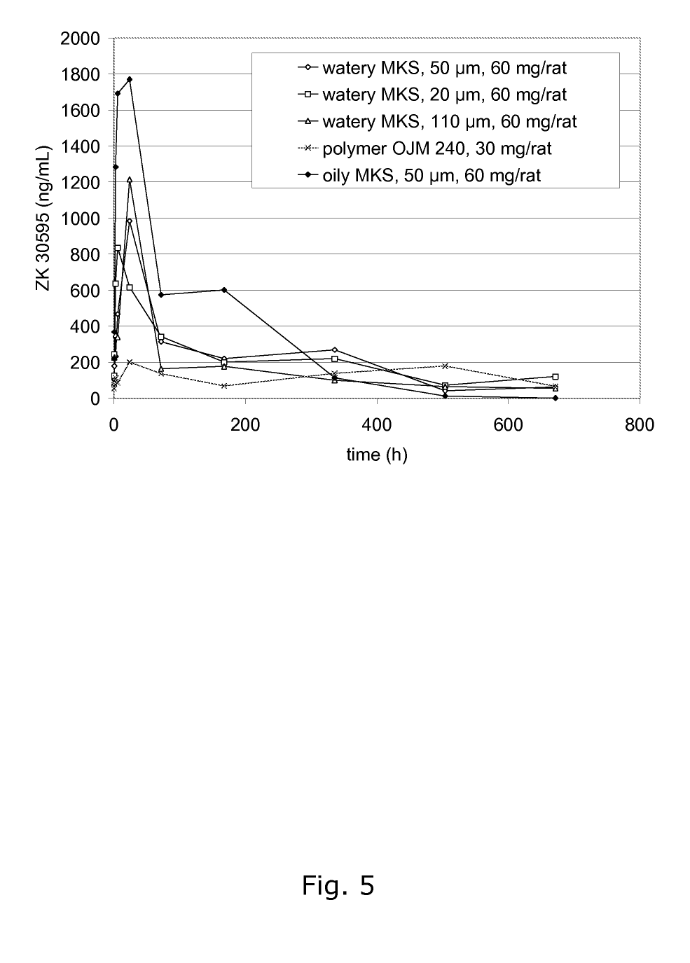

The inventors of the present invention surprisingly found that polyglycolic acid, polylactic acid, poly caprolactones, poly anhydrides and any copolymer of these, e.g. copolymers of glycolic acid and lactic acid could be used to create particles encapsulating Drospirenone that are suitable for subcutaneous administration of Drospirenone. The reason that it was a surprise is two-fold. Firstly, these polymers are short-chain polymers and such polymers are generally known to be useful for short-term kinetics, however the inventors of the present invention found that when they were used for encapsulation of Drospirenone they were able to provide a long-term release profile (FIG. 5).

Secondly, the presence of glycolic and/or lactic acid in the polymers creates an acidic environment inside the particle and Drospirenone is unstable under acidic conditions. It was therefore a surprise to find that Drospirenone could be encapsulated in one of these polymers without notably affecting its stability.

The advantage of using polyglycolic acid, polylactic acid, poly caprolactones, poly anhydrides and any copolymer of these, such as poly(lactide-co-glycolide) copolymers is that these polymers are biocompatible and biodegradable. Poly(lactide-co-glycolide) (PLGA) undergoes hydrolysis in the body to produce the original monomers, lactic acid and glycolic acid. These two monomers under normal physiological conditions, are by-products of various metabolic pathways in the body and they can be metabolised via the tricarboxylic acid cycle.

Polyglycolic acid (PGA) which is also known as polyglycolide or 1,4-Dioxane-2,5-dione is a polymer of the monomer glycolic acid. PGA is a biodegradable, thermoplastic polymer and the simplest linear, aliphatic polyester. Methods of preparing polyglycolic acid are known in the art and include the following processes starting with different materials:
polycondensation of glycolic acid;
ring-opening polymerization of glycolide;
solid-state polycondensation of halogenoacetates;
acid catalyzed reaction of carbon monoxide and formaldehyde

Polylactic acid (PLA) which is also known as polylactide is a polymer of the monomer lactic acid. Due to the chiral nature of lactic acid both D- and L-enantiomers of lactic acid exist and polymers comprising only one of the enantiomers or a combination of both enantiomers exist. It is contemplated that any of these polylactic acid polymers may be used in the present invention; i.e. polymers of the L-enantiomer of lactic acid, polymers of the D-enantiomer of lactic acid or polymers of the L-and D-enantiomers of lactic acid may be used in the present invention.

Polylactic acid may be derived from renewable resources, such as corn starch (in the U.S.) or sugarcanes (rest of world).

Lactic acid is typically produced by bacterial fermentation of corn starch or cane sugar. However, lactic acid cannot be directly polymerized to a useful product, because each polymerization reaction generates one molecule of water, the presence of which degrades the forming polymer chain to the point that only very low molecular weights are observed. Instead, lactic acid is oligomerized and then catalytically dimerized to make the cyclic lactide monomer. Although dimerization also generates water, it can be separated prior to polymerization. PLA of high molecular weight is produced from the lactide monomer by ring-opening polymerization using most commonly a stannous octoate catalyst, but for laboratory demonstrations tin(II) chloride is often employed. This mechanism does not generate additional water, and hence, a wide range of molecular weights are accessible.

The ratio of D-and L-enantiomers in the polymer may be controlled by using stereospecific catalysts. Furthermore, the ratio of D to L enantiomers in the polymer also affects the crystallinity of the polymer.

Thus the polylactic acid polymer of the present invention may be poly(D-lactide), poly(L-lactide) or poly(D,L-lactide). In a particular embodiment it may be poly(D,L-lactide).

The copolymers of PGA and PLA used in the present invention may in particular be copolymers of PGA and PLA which are known as poly(lactic-co-glycolic acid) or PLGA.

PLGA is a copolymer is a biodegradable and biocompatible polymer which is synthesized by means of random ring-opening co-polymerization of two different monomers, the cyclic dimers (1,4-dioxane-2,5-diones) of glycolic acid and lactic acid. Common catalysts used in the preparation of this polymer include tin(II) 2-ethylhexanoate, tin(II) alkoxides, or aluminum isopropoxide. During polymerization, successive monomeric units (of glycolic or lactic acid) are linked together in PLGA by ester linkages, thus yielding a linear, aliphatic polyester as a product.

Depending on the ratio of lactide to glycolide used for the polymerization, different forms of PLGA can be obtained: these are usually identified in regard to the monomers' ratio used (e.g. PLGA 75:25 identifies a copolymer whose composition is 75% lactic acid and 25% glycolic acid. Due to the chiral nature of lactic acid as described PLGA polymers may be either poly(D,L-lactide-co-glycolide), poly(D-lactide-co-glycolide) or poly(L-lactide-co-glycolide) polymers and any of these polymers may be used in the present invention.

In a particular embodiment the ratio of the poly(lactide-co-glycolide) polymer is 50:50, 65:35; 75:25 or 85:15.

The time for degradation of these polymers in vivo is depending on among other things the ratio of lactic acid to glycolic acid and the length of the polymer. In general the higher the ratio of lactic acid to glycolic acid and the longer the polymer the longer it takes to degrade these polymers in vivo. Thus by controlling these parameters a person skilled in the art is able to adjust the degradation half-live of the particle of the present invention.

Examples of commercially available PLGA polymers include are the PLGA Resomer from Boehringer-Ingelheim, such as the PLGA Resomer RG 502 H, PLGA Resomer RG 503 H, PLGA Resomer RG 752 S, PLGA Resomer RG 756 S, PLGA Resomer RG 503 H.

Besides the PLGA copolymers it is envisioned that other copolymers of PGA may be used in the present invention.

For example suitable PGA copolymers include but are not limited to poly(glycolide-co-caprolactone) with ε-caprolactone, and poly(glycolide-co-trimethylene carbonate) with trimethylene carbonate.

Another example of suitable PLGA or PLA polymers include those sold under the name of PURASORB from company PURAC, a division of CSM (Netherlands).

In a particular embodiment of the present invention the Drospirenone may be encapsulated in a polymer selected from the group consisting of poly(D,L-lactide), poly(D,L-lactide-co-glycolide) polymer, poly(D-lactide-co-glycolide) polymer and poly(L-lactide-co-glycolide) polymer.

In a particular embodiment the ratio of Drospirenone and said polymer in the particles of the present invention are in the range of 2:1 to 1:2.

In another embodiment the particles of the present invention comprise 5-50 w/w % Drospirenone.

It is envisioned that the particles of the present invention may comprise other ingredients such as one or more other active ingredients, e.g. steroids, or inactive ingredients, such as ingredients for control of drug release or for cryoprotection.

### Composition

The present invention also relates to a composition comprising particles according to the present invention.

The composition according to the present invention may in particular be fluid or liquid with the particles suspended therein. The particles may for example be suspended in aqueous diluents such as mixture of water/PVA or water/natrium carboxycellulose or water/Tween 20 or 80 in concentration range 0.1-10%.

The composition may comprise other components than the particles of the present invention.

In a particular embodiment the composition of the present invention may comprise a hydrophilic polymer capable of ensuring an adequate stability of the composition. In the context of the present invention the term “hydrophilic” refers to the general understanding of this term thus it refers to the physico-chemical property of molecule or material that can bond water through chemical interaction with H₂O molecule (H-bonding). The hydrophilic polymers which may An example of a group of compounds which may be used as a hydrophilic polymer in the present invention are water soluble celluloses and starches such as carboxymethyl cellulose, methyl cellulose, ethyl cellulose, hydroxymethyl cellulose, hydroxyethyl cellulose, hydroxypropyl cellulose, hydroxymethylpropyl cellulose, hydroxypropylmethyl cellulose and combinations thereof. Particularly preferred cellulosic materials are hydroxypropylmethyl cellulose and hydroxypropyl cellulose, in particular hydroxypropylmethyl cellulose.

Another group of suitable hydrophilic polymers which are useful in the context of the present invention are synthetic polymers, such as polyvinyl alcohol, polyethyleneglycol, polyethylene oxids, or polyvinyl alcohol polyethylene glycol (PVA-PEG) copolymer, or copolymers of any of these.

As described above Drospirenone is often used in combination with e.g. an estrogen which may be encapsulated in the particles of the present invention together Drospirenone or it may be combined or mixed with such particles. Hence in a preferred embodiment the composition of the present invention may further comprise an estrogen, natural or a synthetic derivative thereof. Examples of suitable estrogens which may be present include any of those described above in relation to the particles.

The concentration of Drospirenone in the composition of the present invention may in particular be in the range of 1-250 mg per 1 ml of composition.

### Manufacture

In a further embodiment the present invention relates to a method of producing particles according to the present invention, wherein said method comprises the steps of:

a) mixing Drospirenone and polymer selected from the group consisting of polyglycolic acid, polylactic acid, poly caprolactones, poly anhydrides and any copolymer of these, e.g. copolymers of glycolic acid and lactic acid, in a solvent,

b) adding a hydrophilic polymer to the mixture obtained from step a),

c) emulsifying the mixture obtained from step b) with aqueous phase

d) homogenising,the obtained emulsion from step c)

e) evaporating the solvent from step a).

f) washing, centrifugation or filtration and freeze-drying of obtained suspension after step e)

The Drospirenone and the polymers used in step a) included all of those described above in relation to the particle of the present invention.

In one embodiment of the method the solvent used in step a) is dichloromethane or a mixture of dichloromethane and methanol. Another example of a suitable solvent is ethyl acetate.

Methods of emulsifying or homogenising the mixture as in step c) and d) of the present method are known to a person skilled in the art and may in particular be performed as described in the examples. Examples 5-8 were manufactured by two-step homogenization process.

Filtration is a step before the freeze-drying process in order to reduce amount of water in the suspension. See corrected manufacturing procedure above.

Furthermore, and in particular if the obtained particles are to be stored for a long period of time the particles should be freeze-dried. Advantages of freeze-drying the particles is that the stability of the particles is higher, i.e. it is increased and it also makes handling of the product easier including dosage of the product.

### Use of the Particles and Composition

The present invention also relates to use of the particles and/or composition according to the present invention as a contraceptive; e.g. for inhibition of ovulation in a women and for the treatment of diseases, disorders and symptoms associated with deficient endogenous levels of estrogen in women.

Hence the present invention also relates particles or a composition according to the present invention for the use as a medicament. In a particular embodiment such use may for subcutaneous administration.

The present invention also relates to a method for inhibition of ovulation in a women comprising subcutaneous injection of the particles or a composition according to the present invention. In a particular embodiment said particles or said composition is administered once every 24-30 days.

The present invention also relates to a method for the treatment of diseases, disorders and symptoms associated with deficient endogenous levels of estrogen in a women comprising subcutaneous injection of the particles or a composition according to the present invention.

In the present context, the term cycle itself or when associated with the term menstrual is intended to mean the number of days between menses in a woman. It can range from 21-31 days, typically 28 days.

In the present context, the term menopause is understood as the last natural (ovary-induced) menstruation. It is a single event and a result of an age-dependent dysfunction of the ovarian follicles. Menopause results from the ovaries decreasing their production of the sex hormones estrogen and progesterone. When the number of follicles falls below a certain threshold (a bleeding threshold), the ovaries can no longer produce mature follicles and sex hormones. The ability to reproduce capability ends with menopause.

The peri-menopausal phase begins with the onset of climacteric symptoms when the cycle becomes irregular and ends one year after menopause. The end of peri-menopausal phase can be identified after a protracted period of time without bleeding. Post-menopause is the phase that begins at menopause and continues until death.

One principal aim of hormone replacement therapy is to restore levels of the sex steroid hormones in naturally or prematurely pre-menopausal, menopausal and post-menopausal women or to establish these levels in hypogonadal females.

Deficient levels of estrogen can occur for a variety of reasons. The composition can be such that it is adequate for deficient levels of estrogen, regardless of the cause. Causes anticipated by the therapy are, but not limited to, natural menopause, peri-menopause, post-menopause, hypogonadism, castration or primary ovarian failure.

Low levels of estrogen, irrespective of the cause, lead to an overall decreased quality of life for women. Symptoms, diseases and disorders range from merely being inconvenient to life threatening. The composition of this therapy anticipates the effective alleviation of all physiological and psychological signs of estrogen deficiency.

Transient symptoms, such as vasomotor signs and psychological symptoms are certainly embodied with the realm of therapy. Vasomotor signs comprise but are not limited to hot flushes, sweating attacks such as night sweats, and palpitations. Psychological symptoms of estrogen deficiency comprise, but are not limited to, insomnia and other sleep disorders, poor memory, loss of confidence, mood changes, anxiety, loss of libido, difficulties in concentration, difficulty in making decisions, diminished energy and drive, irritability, and crying spells.

The treatment of the aforementioned symptoms can be associated with the peri-menopausal phase of a woman's life or after, sometimes long after menopause. It is anticipated that the invention is applicable to these and other transient symptoms during the peri-menopausal phase, menopause, or post-menopausal phase. Moreover, the aforementioned symptoms can be alleviated if the cause of the estrogen deficiency is hypogonadism, castration or primary ovarian failure.

In another embodiment of the invention, the therapy is used for the treatment of permanent effects of estrogen deficiency. Permanent effects comprise physical changes such as urogenital atrophy, atrophy of the breasts, cardiovascular disease, changes in hair distribution, thickness of hair, changes in skin condition and osteoporosis.

Urogenital atrophy, conditions associated with it such as vaginal dryness, increase in vaginal pH and subsequent changes in flora, or events which lead to such atrophy, such as decreases in vascularity, fragmentation of elastic fibres, fusion of collagen fibres, or decreases in cell volume are symptoms thought to be particularly relevant to this therapy. Furthermore, the invention is thought to be relevant to other urogenital changes associated estrogen deficiency such as decreases in the length and/or diameter of the vagina, decreases mucus production, changes in cell population, decreases in glycogen production, decreases in growth of lactobacilli or increases in growth of streptococci, staphylococci, or coliform bacilli. Other associated changes that are thought to be preventable by the invention are those that may render the vagina susceptible to injury or infection, such as exudative discharges, vaginitis, and dyspareunia. Furthermore, infections of the urinary tract and incontinence are other common symptoms associated with lowered estrogen levels.

Other embodiments of the invention include the prevention or alleviation of physical changes associated with estrogen deficiency, such as changes in the skin, changes in hair distribution, thickness of hair, atrophy of the breasts, or osteoporosis.

The prevention and management of osteoporosis, most notably post-menopausal osteoporosis, is a particularly interesting embodiment of the invention. Furthermore, bone demineralisation, reduction of bone mass and density, thinning and interruption of trabeculae, and/or consequent increase in bone fractures or bone deformations are thought to be particularly relevant. The prophylactic treatment of osteoporosis is an interesting therapeutic application of the invention.

A particularly interesting embodiment of the invention comprises the use of the composition for lessening the frequency, persistence, duration and/or severity of hot flushes, sweating attacks, palpitations, sleep disorders, mood changes, nervousness, anxiety, poor memory, loss of confidence, loss of libido, poor concentration, diminished energy, diminished drive, irritability, urogenital atrophy, atrophy of the breasts, cardiovascular disease, changes in hair distribution, thickness of hair, changes in skin condition and osteoporosis, most notably hot flushes, sweating attacks, palpitations, sleep disorders, mood changes, nervousness, anxiety, urogenital atrophy, atrophy of the breasts or for the prevention or management of osteoporosis.

The particles and/or the composition according to the present invention may in particular be administered by injection. Typically it is a composition according to the present invention which is used in practice. The term “administration by injection” is meant to encompass any form for injection into a muscle or subcutaneous injection. The preferred form of injection is by subcutaneous injection.

The volume that can be injected intramuscularly is known to affect the release rate of an active principle from a vehicle. An injection volume of 1 mL is generally considered as the maximum volume that can be administered by on single subcutaneous injection to one injection. Similarly, the maximum injection intramuscular injection volume is generally considered to be 5 mL When the injection of volumes greater than those maximum values is required, the injection volume needs to be divided into two or more separate injections to different injection sites. However, multiple injections for the administering of one dose are generally not preferred because of the inconvenience conferred to the female.

Furthermore, the injection of a single dose to one injection site offers great advantages in controlling the release rate of an active principle, rather than multiple injection of divided single doses. Thus in interesting embodiments the subcutaneous injection volume is typically in the range of 0.2-1 mL, while the intramuscular injection volume is typically in the range of 1-5 mL.

The composition may be suitable formulated as a unit dose form such as a unit dose intended for being injected as one single dose.

Independent of whether composition of the present invention is administered subcutaneously or intramuscularly the injected single dose of Drospirenone may typically be in the range of 1-250 mg, such as between 30-200 mg of Drospirenone.

The compositions may typically be administered once during the menstrual cycle, such as once every 24-30 days.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Materials and Methods

All PLGA polymers were PLGA polymer Resomer® obtained from Boehringer Ingelheim.

Drospirenone is an in-house product and characterized by internal documents.

### Example 1

### Encapsulation of Drospirenone in PLGA Polymer Comprising Lactic Acid and Glycolic Acid Monomers

Preparation of Micro Particles

Two different methods for preparation of the micro particles were used, one including one-step homogenisation and the other including two-step homogenisation. Emulsions 1-4 were prepared by the method including one-step homogenisation and emulsions 5-8 were prepared by the method including two-step homogenisation.

Method with One-step Homogenisation

The commercially available PLGA polymer Resomer® RG 503H from Boehringer Ingelheim was used in the present example.

PLGA polymer and Drospirenone were mixed with a mixture of dichloromethane (DCM) and methanol (MeOH) in a 10 ml vial before adding it to a 0.4% polyvinylalcohol (PVA) 4-88 solution in a 100 ml beaker.

The mixture was then emulsified for 3 hours at 500 rpm (Rührer RCT Basic, Ika, Deutschland) before adding it to 800 ml Millipore water in a 1000 ml beaker (Fischerbrand, UK) at 400 rpm. Subsequently the mixture was stirred for 3 hours at 400 rpm to evaporate the dichloromethane.

The suspension with the micro particles was then filtered onto at Whatman Filter 6 using a suction strainer. The obtained filter cake was subsequently transferred and divided into two vials before each of them were resuspended in 5 ml water and freeze-dried (Ismatec, Deutschland).

Four emulsions 1-4 comprising different amounts of the components were used to create micro particles according to this procedure. The contents of these four emulsions are shown in table 1 below.

TABLE 1

| | Emul- | | Emul- | | Emul- | | Emul- | |
|---|---|---|---|---|---|---|---|---|
| | sion 1 | | sion 2 | | sion 3 | | sion 4 | |
| PLGA RG 503H | 100 | mg | 75 | mg | 100 | mg | 75 | mg |
| (mg) | | | | | | | | |
| Drospirenone (mg) | 150 | mg | 175 | mg | 150 | mg | 175 | mg |
| Dichloromethane | 2.5 | ml | 2.5 | ml | 2.25 | ml | 2.25 | ml |
| (ml) | | | | | | | | |
| Methanol (ml) | 0 | ml | 0 | ml | 0.25 | ml | 0.25 | ml |
| 0.4% PVA (ml) | 60 | ml | 60 | ml | 60 | ml | 60 | ml |

Method with Two-step Homogenisation

The commercially available PLGA polymer Resomer® RG 502H, RG 503H, RG 752S and RG 756S from Boehringer Ingelheim were used in the present example. PLGA polymer and Drospirenone were dissolved in dichloromethane (DCM) in a 10 ml vial and in a 1000 ml beaker (Fischerbrand, UK) 0.25% polyvinylalcohol (PVA) 40-88 solution was injected under the surface to the solution. The emulsion was stirred at 2000 rpm for 3 min (Rührer RCT Basic, Ika, Deutschland).

The emulsion was then left for 3 min prior to homogenising it with Ultra-Turrax T25 Basis Homogeniser (Ika, Deutschland) for 3 min at 9500 rpm. Subsequently the suspension was stirred for 2 hours at 400 rpm to evaporate the dichloromethane.

The suspension with the micro particles was then filtered onto at Whatman Filter 6 using a suction strainer. The obtained filter cake was subsequently transferred and divided into two vials before each of them were resuspended in 5 ml water and freeze-dried (Ismatec, Deutschland).

Four emulsions 5-8 comprising different amounts of the components were used to create micro particles according to this procedure. The contents of these four emulsions are shown in table 2 below.

TABLE 2

| | Emul- | Emul- | Emul- | Emul- |
|---|---|---|---|---|
| | sion 5 | sion 6 | sion 7 | sion 8 |
| PLGA RG 502H (mg) | 175 | 0 | 0 | 0 |
| PLGA RG 503H (mg) | 0 | 175 | 175 | 350 |
| PLGA RG 752S (mg) | 0 | 0 | 175 | 0 |
| PLGA RG 756S (mg) | 175 | 175 | 0 | 0 |
| Drospirenone (mg) | 245 | 245 | 245 | 245 |
| Dichloromethane | 3 | 3 | 3 | 3 |
| (ml) | | | | |
| 0.25% PVA 40-88 | 800 | 800 | 800 | 800 |
| (ml) | | | | |

Characterisation of the Micro Particles

The size of the micro particles was determined by a laser diffraction particle size analyzer Beckmann Coulter LS 13 320. A sample of the micro particles was injected in a suspension of distilled water with a micro particle concentration of 10-20 mg/ml. The apparatus was automatically calibrated.

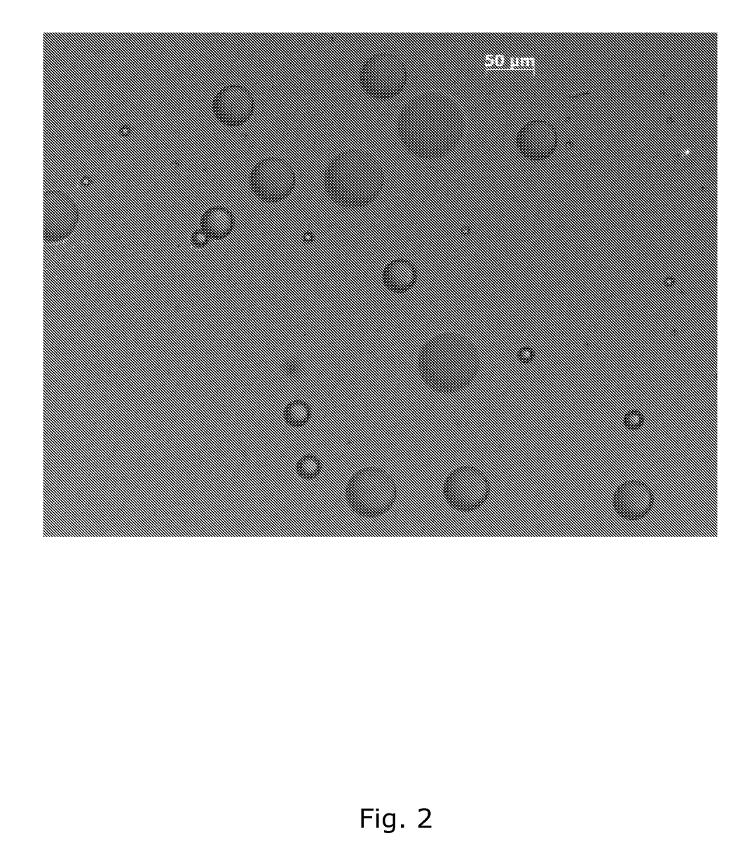

The shape and the surface of the micro particles was investigated with a light microscope and an example of the micro particles formed using emulsion 1 is shown in FIG. 1 and the micro particles formed using emulsion 8 is shown in FIG. 2.

The amount of Drospirenone in the micro particles was determined with HPLC Waters 2695 with Waters UV Detector 2487. The retention time for Drospirenone was 2.7 min and it was detected at 270 nm wavelength which had been determined with a Drospirenone standard prior to detection of the Drospirenone in the micro particles. The amount of Drospirenone was measured for three samples of each of the four emulsions and the results are shown in tables 3 and 4 below.

TABLE 3

| | Emulsion 1 | Emulsion 2 | Emulsion 3 | Emulsion 4 |
|---|---|---|---|---|
| Size d₅₀ (μm) | 69.46 μm | 63.21 μm | 40.86 μm | 38.44 μm |
| Drospirenone | 31.7% (w/w) | 24.4% (w/w) | 22.9% (w/w) | 24.4% (w/w) |
| content (% (w/w)) | | | | |

TABLE 4

| | Emulsion 5 | Emulsion 6 | Emulsion 7 | Emulsion 8 |
|---|---|---|---|---|
| Size d₅₀ (μm) | 58.55 μm | 67.39 μm | 122.4 μm | 55.17 μm |
| Drospirenone | 30.1% (w/w) | 34.6% (w/w) | 32.9% (w/w) | 33.2% (w/w) |
| content (% (w/w)) | | | | |

In Vitro Release Study

Samples of the above mentioned emulsions 1-8 containing each 20 mg of freeze-dried micro particles were added to each 15 ml of Release medium (Sörensen's phosphate-buffer pH 7.4, 5% Tween 20) and the mixtures were shaken at 37° C. (shaker Innova 4230, New Brunswich Scientific, USA). At the given time points 1000 μl samples were withdrawn to measure the amount of Drospirenone released. The samples were centrifuged for 7 minutes at 9000 rpm (Sigma 2K15 Laboratory centrifuge) and 800 μl of the supernatant was used to determine the amount of Drospirenone with HPLC as described above.

The precipitate was resuspended in 800 μl Release medium and returned to the release study.

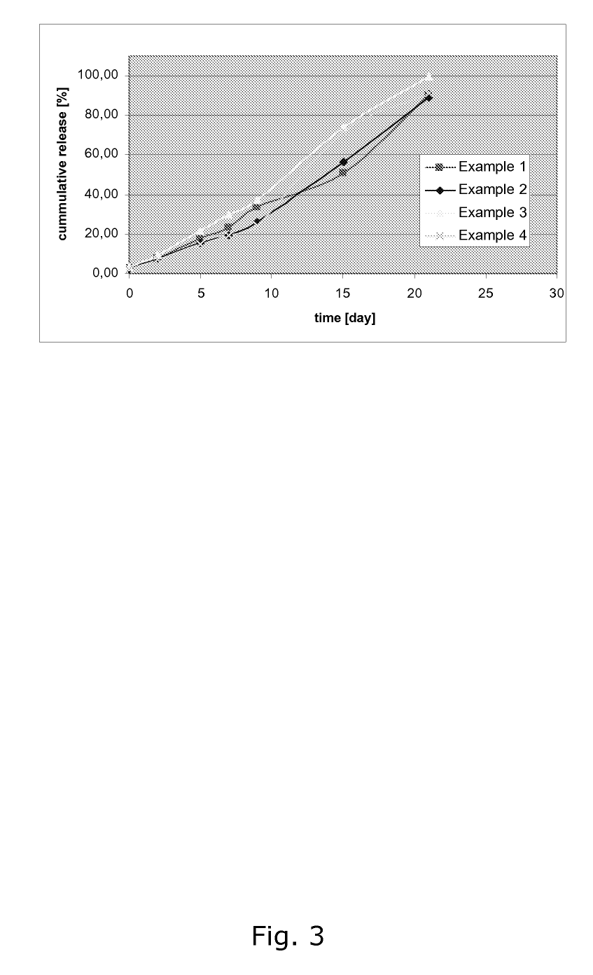

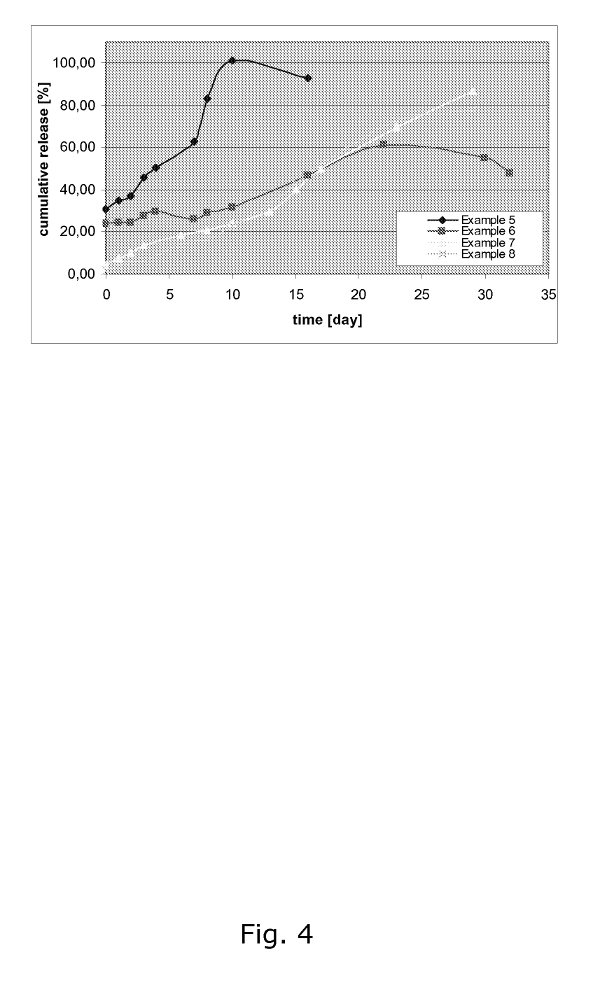

The results are shown in FIG. 3 for emulsions 1-4 and FIG. 4 for emulsions 5-8. The examples referred to therein correspond to the same emulsions.

### Example 2

### In Vivo Pharmacokinetics of Drospirenone Encapsulated in Polymer Compromising Lactic Acid and Glycolic Acid

Study Objectives & Description

The aim of the present study was to evaluate the release of Drospirenone from subcutaneous depot formulations in the rat. The present study was designed as an open, non-randomized comparison between various depot formulations containing DRSP—derivatives used for contraception (Table 4). Pharmacokinetic parameters were calculated from the mean serum concentration-time profiles.

TABLE 4
Overview of treatments and sample origin

| | Formulation | Actual Dose | Sampling times |
|---|---|---|---|
| Analyte | [group] | [mg/rat] | [h] |
| Drospirenone | Aqueous MKS | 60 | 0.5, 1, 3, 6, 24, 72, |
| | (50 μm, group 1) | | 168, 336, 504, 672 |
| | Aqueous MKS | 60 | 0.5, 1, 3, 6, 24, 72, |
| | (20 μm, group 5) | | 168, 336, 504, 672 |
| | Aqueous MKS | 60 | 0.5, 1, 3, 6, 24, 72, |
| | (110 μm, group 6) | | 168, 336, 504, 672 |
| | PLGA Particles | 30 | 0.5, 1, 3, 6, 24, 72, |
| | (group 9) | | 168, 336, 504, 672 |
| | Oily MKS | 60 | 0.5, 1, 3, 6, 24, 72, |
| | (50 μm, group12) | | 168, 336, 504, 672 |

The PLGA particles type Emulsion 1 were prepared as described above.

The aqueous microcrystalline suspension was prepared as follows:

Drospirinone (micronized, 20, 50 or 110 μm respectively) was suspended using a magnetic stirrer (Ika-Werke, RCT, Germany) in isotonic NaCl solution containing 0.25% Tween 80 and 1% Klucel LF.

The oily suspension of Drospirenone was prepared as follows: 5.4 mg Drospirenone (micronized, size 50 μm) was added to 45.0 ml peanut oil. The suspension was been blended with a magnetic stirrer (Ika-Werke, RCT, Germany)

### Summary of Results and Discussion

The mean pharmacokinetic parameters are summarized in FIG. 5 (Mean serum concentrations of Drospirenone after single subcutaneous administration of Drospirenone in various depot formulations).

In contrast to the other applied formulations, the pharmacokinetic data of encapsulated Drospirenone showed a constant drug release over 28 days without significant burst effect within first 48 hours.

1. A particle comprising Drospirenone encapsulated in a polymer selected from the group consisting of polyglycolic acid, polylactic acid, poly caprolactones, poly anhydrides and any copolymer of these.

2. The particle according to claim 1, wherein the polymer is selected from the group consisting of: poly(D,L-lactide), poly(D,L-lactide-co-glycolide) polymer, poly(D-lactide-co-glycolide) polymer and poly(L-lactide-co-glycolide) polymer.

3. The particle according to claim 2, wherein the ratio of the poly(lactide-co-glycolide) polymer is 50:50, 65:35; 75:25 or 85:15.

4. The particle according to claim 1, wherein the ratio of Drospirenone and said polymer is in the range of 2:1 to 1:2.

5. The particle according to claim 1, wherein the d50 of the particle is in the range of 10-200 μm.

6. The particle according to claim 1, comprising between 5-50 w/w % Drospirenone.

7. The particle according to claim 1, wherein said particles further comprise an estrogen encapsulated in the polymer.

8. A pharmaceutical composition comprising a plurality of the particle according to claim 1 together with an excipient.

9. A composition according to claim 8, wherein the concentration of Drospirenone is in the range of 1-250 mg per 1 ml composition.

10. A composition according to claim 8, wherein said composition further comprises an estrogen.

11. A method of producing the particle according to claim 1 comprising the steps of
a) mixing Drospirenone and a polymer selected from the group consisting of polyglycolic acid, polylactic acid, poly caprolactones, poly anhydrides and any copolymer of these, in a solvent,
b) adding a hydrophilic polymer to the mixture obtained from step a),
c) emulsifying the mixture obtained from step b) with an aqueous phase,
d) homogenizing the obtained emulsion from step c),
e) evaporating the solvent from step a), and
f) washing, centrifugation or filtration and freeze-drying of obtained suspension after step e).

12. A method according to claim 11, wherein the solvent is dichloromethane or a mixture of dichloromethane and methanol or ethyl acetate.

13-14. (canceled)

15. A method for inhibition ovulation in a woman comprising the step of administering as a subcutaneous injection a composition comprising a plurality of the particle according to claim 1 together with an excipient.

16. A method according to claim 13, wherein the composition is administered once every 24-30 days.

17. The composition according to claim 8 adapted for subcutaneous administration.
